(19) United States
(12) Patent Application Publication
(10) Pub. No.: US 2025/0011430 A1
(43) Pub. Date: Jan. 9, 2025

(54) ANTI-B7H3 ANTIBODY AND USE THEREOF

(71) Applicant: GUANGZHOU BIO-GENE TECHNOLOGY CO., LTD, Guangdong (CN)

(72) Inventors: Min Luo, Guangdong (CN); Guangchao Li, Guangdong (CN); Zhao Zhou, Guangdong (CN); Xuejun Wang, Guangdong (CN)

(21) Appl. No.: 18/003,996

(22) PCT Filed: Dec. 15, 2020
(86) PCT No.: PCT/CN2020/136409

§ 371 Date: Dec. 30, 2022

(30) Foreign Application Priority Data
Jun. 30, 2020 (CN) 202010622482.X

(51) Int. Cl. C07K 16/28; A61K 39/00; C12N 15/63

(52) U.S. Cl. CPC C07K 16/2827; C12N 15/63; A61K 2039/505; C07K 2317/41; C07K 2317/52; C07K 2317/565

(57) ABSTRACT

Provided are an anti-B7H3 antibody and the use thereof. A variable region of the antibody comprises amino acid sequences as shown in SEQ ID NOs: 25-39. The anti-B7H3 antibodies 26B6, 6F7, 2B8, and 23H1 have a significant binding capability to B7H3, can not only bind to purified or free B7H3 protein, but also can bind to B7H3 protein on a cell surface; and after humanized modification, the affinity between the antibody and B7H3 is further improved, and the antibody has important application prospects in clinical diagnosis and/or treatment of B7H3-positive tumors.

### TECHNICAL FIELD

The present application belongs to the field of biomedical technology and relates to an anti-B7H3 antibody and a use thereof.

### BACKGROUND

B7-H3 is a type I transmembrane protein, which belongs to a B7 immune co-stimulation and co-suppression family with two isotypes 2Ig-B7-H3 and 4Ig-B7-H3 in humans and one isotype 2Ig-B7-H3 in mice, where human 2Ig-B7-H3 has an 88% of amino acid identity with mouse 2Ig-B7-H3. B7-H3 has an immunosuppressive function, which can reduce type I interferon (IFN) released from T cells and reduce cytotoxicity of NK cells.

B7-H3 proteins have limited expression in normal tissues (such as prostate, breast, placenta, liver, colon and lymphoid organs) but are abnormally expressed in most malignant tumors. B7-H3 expression can be detected in non-small-cell lung cancer cell lines and tumor tissues. B7-H3 mRNA and B7-H3 proteins are highly expressed in all six non-small-cell lung cancer cell lines and also expressed in a cell membrane and cytoplasm of cancer cells with an expression rate of about 73%. In the tumor tissues expressing B7-H3, the number of infiltrating lymphoid cells is significantly reduced and positively correlated with lymph node metastasis (Sun Y, Wang Y, Zhao J, et al. B7-H3 and B7-H4 expression in non-small-cell lung cancer[J]. Lung Cancer, 2006, 53(2): 143-151; Zhao Wenjian, Chen Chunyan, Sui Wenyan, et al. Advances in B7-H3 and its relationship with tumors[J]. Medical Recapitulate, 2009, 15(22): 3430-3433.).

Generally, B7-H3 overexpression in tumor cells is closely associated with reduced tumor-infiltrating lymphocytes, accelerated cancer progression and clinical outcomes of malignant tumors (nervous system tumor, melanoma, lung cancer, head and neck cancer, colorectal cancer, pancreatic cancer, prostate cancer, ovarian cancer, lung cancer and clear cell renal cell carcinoma). B7-H3 has become a potential target for cancer immunotherapy due to widespread expression in a variety of tumors. A B7-H3-specific monoclonal antibody (mAb) and an antibody-drug conjugate show anti-tumor activity against B7-H3-positive tumor cells in mouse xenograft models, and a phase I clinical trial shows a good safety profile (NCT01099644, NCT02381314 and NCT02982941). Since B7-H3 is highly expressed in tumor histiocytes but not or low expressed in normal histiocytes, B7-H3 is considered as a diagnostic marker for certain tumors. B7-H3 is closely related to biological characteristics of tumors and has become one of the hot targets for the treatment of malignant tumors.

A structure of human B7H3 is shown in FIG. 1. An extracellular structure of B7H3 is composed of two Ig-V domain+Ig-C2 domain in tandem with an almost identical sequence. A highly expressed alternative spliceosome lacks an intermediate moiety, and an extracellular region has only one Ig-V domain+Ig-C2 domain. Mouse B7H3 has only one IgV+IgC2 unit. A structure of Ig-V domain+Ig-C2 domain is similar to that of PD-L1. The four domains are linked mainly by flexible linker peptides and can swing freely. B7H3 is mainly expressed as a monomer, and homodimerization may occur in vitro. However, no protein with a homology of more than 30% with B7H3 is present in a human body.

### SUMMARY

The present application is to provide an anti-B7H3 antibody and a use thereof. The antibody is used alone and/or in combination with other drugs for the treatment of cancers.

To achieve this, the present application adopts technical solutions described below.

In a first aspect, the present application provides an anti-B7H3 antigen-binding fragment. An amino acid sequence of CDR3 of a heavy chain variable region of the antigen-binding fragment is as shown in SEQ ID NO: 1, SEQ ID NO: 2, SEQ ID NO: 3 or SEQ ID NO: 4;
an amino acid sequence of CDR3 of a light chain variable region of the antigen-binding fragment is as shown in SEQ ID NO: 5, SEQ ID NO: 6, SEQ ID NO: 7 or SEQ ID NO: 8;
an amino acid sequence of CDR2 of the heavy chain variable region of the antigen-binding fragment is as shown in SEQ ID NO: 9, SEQ ID NO: 10, SEQ ID NO: 11 or SEQ ID NO: 12;
an amino acid sequence of CDR2 of the light chain variable region of the antigen-binding fragment is as shown in SEQ ID NO: 13, SEQ ID NO: 14, SEQ ID NO: 15 or SEQ ID NO: 16;
an amino acid sequence of CDR1 of the heavy chain variable region of the antigen-binding fragment is as shown in SEQ ID NO: 17, SEQ ID NO: 18, SEQ ID NO: 19 or SEQ ID NO: 20;
an amino acid sequence of CDR1 of the light chain variable region of the antigen-binding fragment is as shown in SEQ ID NO: 21, SEQ ID NO: 22, SEQ ID NO: 23 or SEQ ID NO: 24. Unless otherwise stated, positions of specific amino acid residues in an antibody variable region are numbered according to a Kabat numbering system.

According to the present application, a heavy chain variable region of an antigen-binding fragment of an anti-B7H3 antibody 26B6 includes CDR1 as shown in SEQ ID NO: 17, CDR2 as shown in SEQ ID NO: 9 and CDR3 as shown in SEQ ID NO: 1;
a light chain variable region includes CDR1 as shown in SEQ ID NO: 21, CDR2 as shown in SEQ ID NO: 13 and CDR3 as shown in SEQ ID NO: 5.

SEQ ID NO: 17:
GYAFTEY;
SEQ ID NO: 9:
NPNTGG;
SEQ ID NO: 1:
PYRDDGGFHWYFDV;
SEQ ID NO: 21:
SASSSVSYMQ;
SEQ ID NO: 13:
DTSKLTS;
SEQ ID NO: 5:
QQWSSNPLT.

The heavy chain variable region of the anti-B7H3 antibody 26B6 containing the above antigen-binding fragments includes an amino acid sequence as shown in SEQ ID NO: 25, and the light chain variable region includes an amino acid sequence as shown in SEQ ID NO: 26. A heavy chain variable region of humanized 26B6 includes an amino acid sequence as shown in SEQ ID NO: 27, and a light chain variable region includes an amino acid sequence as shown in SEQ ID NO: 28.

SEQ ID NO: 25 (26B6HV):
EVQLQQSGPELVKPGASVKISCKTSGYAFTEYTMHWVKQSQGKSLEWIG
GINPNTGGTTYNQKFNGKATLTVDRSSSTAYMELRSLTSEDSAVYYCTR
PYRDDGGFHWYFDVWGAGTAVTVSSAS;
SEQ ID NO: 26 (26B6LV):
QIVLTQSPAVMSTSPGEKVTMTCSASSSVSYMQWYQQKSGTSPKRWIYD
TSKLTSGVPARFSGSGSGTSYSLTISSMEAEDAATYYCQQWSSNPLTFG
AGTKLELKRADAAP;
SEQ ID NO: 27 (hz26B6HV):
EVQLVQSGAEVKKPGASVKVSCKASGYTFTEYTMHWVRQAPGQGLEWIG
GINPNTGGTTYNQKFNGRVTMTRDTSISTAYMELSSLRSEDTAVYYCTR
PYRDDGGFHWYFDVWGQGTLVTVSS;
SEQ ID NO: 28 (hz26B6LV):
EIVLTQSPATLSLSPGERATLSCSASSSVSYMQWYQQKPGLAPRLLIYD
TSKLTSGIPDRFSGSGSGTDFTLTISRLEPEDFAVYYCQQWSSNPLTFG
GGTKVEIKRTV.

According to the present application, a heavy chain variable region of an antigen-binding fragment of an anti-B7H3 antibody 6F7 includes CDR1 as shown in SEQ ID NO: 18, CDR2 as shown in SEQ ID NO: 10 and CDR3 as shown in SEQ ID NO: 2;
a light chain variable region includes CDR1 as shown in SEQ ID NO: 22, CDR2 as shown in SEQ ID NO: 14 and CDR3 as shown in SEQ ID NO: 6.

SEQ ID NO: 18:
GFTFTDY;
SEQ ID NO: 10:
RNKANGYT;
SEQ ID NO: 2:
DSHYRPFAY;
SEQ ID NO: 22:
KSSQSLLNSGNQNNYLT;
SEQ ID NO: 14:
LASTRDS;
SEQ ID NO: 6:
QNDYTYPLT.

The heavy chain variable region of the anti-B7H3 antibody 6F7 containing the above antigen-binding fragments includes an amino acid sequence as shown in SEQ ID NO: 29, and the light chain variable region includes an amino acid sequence as shown in SEQ ID NO: 30. A heavy chain variable region of humanized 6F7 includes an amino acid sequence as shown in SEQ ID NO: 31, and a light chain variable region includes an amino acid sequence as shown in SEQ ID NO: 32 or SEQ ID NO: 33.

SEQ ID NO: 29 (6F7HV):
EVKLVESGGGLVQPGGSLRLSCATSGFTFTDYYMSWVRQPPGKALEWLG
FIRNKANGYTTEYSASVKGRFTISSDDSQSILYLQMNTLRAEDSATYYC
ARDSHYRPFAYWGQGTLVTVSAAS;
SEQ ID NO: 30 (6F7LV):
DIQMTQSPSSLTVTAGEKVTMSCKSSQSLLNSGNQNNYLTWYQQKPGQP
PKLLIYLASTRDSGVPDRFTGSGSGTDFTLTISSVQAEDLAVYYCQNDY
TYPLTFGAGTKLELKRADAAP;
SEQ ID NO: 31 (hz6F7HV):
EVQLVESGGGLVQPGGSLRLSCAASGFTFTDYYMSWVRQAPGKGLEWVA
FIRNKANGYTTEYSASVKGRFTISRDDSKNSLYLQMNSLRAEDTAVYYC
ARDSHYRPFAYWGQGTLVTVSS;
SEQ ID NO: 32 (hz6F7LV1):
DIVMTQSPLSLPVTPGEPASISCKSSQSLLNSGNQNNYLTWYLQKPGQS
PQLLIYLASTRDSGVPDRFSGSGSGTDFTLKISRVEAEDVGVYYCQNDY
TYPLTFGGGTKVEIKRTV;
SEQ ID NO: 33 (hz6F7LV2):
DIVMTQSPDSLAVSLGERATINCKSSQSLLNSGNQNNYLTWYQQKPGQP
PKLLIYLASTRDSGVPDRFSGSGSGTDFTLTISSLQAEDVAVYYCQNDY
TYPLTFGGGTKVEIKRTV.

According to the present application, a heavy chain variable region of an antigen-binding fragment of an anti-B7H3 antibody 2B8 includes CDR1 as shown in SEQ ID NO: 19, CDR2 as shown in SEQ ID NO: 11 and CDR3 as shown in SEQ ID NO: 3;
a light chain variable region includes CDR1 as shown in SEQ ID NO: 23, CDR2 as shown in SEQ ID NO: 15 and CDR3 as shown in SEQ ID NO: 7.

SEQ ID NO: 19:
GYTFTDG;
SEQ ID NO: 11:
NTNSGN;
SEQ ID NO: 3:
GVFYYGYGAWFAY;
SEQ ID NO: 23:
RASKTISNYLA;
SEQ ID NO: 15:
SGSTLQS;
SEQ ID NO: 7:
QQHHEYPLT.

The heavy chain variable region of the anti-B7H3 antibody 2B8 containing the above antigen-binding fragments includes an amino acid sequence as shown in SEQ ID NO: 34, and the light chain variable region includes an amino acid sequence as shown in SEQ ID NO: 35. A heavy chain variable region of humanized 2B8 includes an amino acid sequence as shown in SEQ ID NO: 36, and a light chain variable region includes an amino acid sequence as shown in SEQ ID NO: 37.

SEQ ID NO: 34 (2B8HV):
QVQLQQSGPELVRPGVSVKISCKVSGYTFTDGAMHWVKRSHAKSLEWIG
IINTNSGNTNYNQKFQGKATMTVDKSSSTAYMELARLTSEDSAIYYCAR
GVFYYGYGAWFAYWGQGTLVTVSAAS;
SEQ ID NO: 35 (2B8LV):
DVQITQSPSYLTASPGETIIINCRASKTISNYLAWYQEKPGKTNKLLIY
SGSTLQSGIPSRFSGSGSDTDFTLTISSLEPEDFAMYYCQQHHEYPLTF
GAGTKLELKRADAAP;
SEQ ID NO: 36 (hz2B8HV):
QVQLVQSGAEVKKPGASVKVSCKVSGYTFTDGAMHWVRQAPGQGLEWIG
IINTNSGNTNYNQKFQGRVTMTRDTSISTAYMELSRLRSEDTAVYYCAR
GVFYYGYGAWFAYWGQGTLVTVSS;
SEQ ID NO: 37 (hz2B8LV):
DIQLTQSPSFLSASVGDRVTINCRASKTISNYLAWYQQKPGKAPKLLIY
SGSTLQSGVPSRFSGSGSGTDFTLTISSLQPEDFATYYCQQHHEYPLTF
GGGTKVEIKRTV.

According to the present application, a heavy chain variable region of an antigen-binding fragment of an anti-B7H3 antibody 23H1 includes CDR1 as shown in SEQ ID NO: 20, CDR2 as shown in SEQ ID NO: 12 and CDR3 as shown in SEQ ID NO: 4;
a light chain variable region includes CDR1 as shown in SEQ ID NO: 24, CDR2 as shown in SEQ ID NO: 16 and CDR3 as shown in SEQ ID NO: 8.

SEQ ID NO: 20:
GFTFTDY;
SEQ ID NO: 12:
RNKVNDYT;
SEQ ID NO: 4:
DSPYRPFAY;
SEQ ID NO: 24:
KSSQTLLNNGNQKNFLT;
SEQ ID NO: 16:
LASTRES;
SEQ ID NO: 8:
NDYTYPLT.

The heavy chain variable region of the anti-B7H3 antibody 23H1 containing the above antigen-binding fragments includes an amino acid sequence as shown in SEQ ID NO: 38, and the light chain variable region includes an amino acid sequence as shown in SEQ ID NO: 39.

SEQ ID NO: 38 (23H1HV):
EVKLVESGGGLVQPGGSLRLSCATSGFTFTDYYMSWVRQPPGKALEWLG
FIRNKVNDYTTEYSVSVKGRFTISRDNSQTILYLQMNTLRAEDSATYYC
ARDSPYRPFAYWGQGTLVTVSAAS;
SEQ ID NO: 39 (23H1LV):
DIVMTQSPSSLTVTAGENVTMSCKSSQTLLNNGNQKNFLTWYQQKPGQP
PKLLIYLASTRESGVPDRFTGSGSGTDFTLTISSVQAEDLAVYYCQNDY
TYPLTFGAGTKLELKRADAAP.

Preferably, the antibody further includes a constant region.

Preferably, an antibody molecule may be an N-terminal, internal or C-terminal modification, such as an oligomerization modification, a glycosylation modification or a modification of conjugation with a marker, thereby adjusting a function of the antibody.

According to the present application, antibody glycosylation can adjust the function of the antibody and affect a half-life, immunogenicity, antibody-dependent cell-mediated cytotoxicity (ADCC) and complement-dependent cytotoxicity (CDC) of the antibody. The antibody glycosylation is mainly related to factors such as an antibody sequence, an amino acid exposure site and a synthesis condition. According to types of glycosidic chains, protein glycosylation may be divided into four types, that is, hydroxyl of serine, threonine, hydroxylysine and hydroxyproline is used as a linkage point to form an —O-glycosidic bond type, an amide group of asparagine, α-amino of an N-terminal amino acid and ω-amino of lysine or arginine are used as linkage points to form an —N-glycosidic bond type, a free carboxyl group of an aspartic acid or glutamate is used as a linkage point to form a lipo-glycosidic bond type; and cysteine is used as a linkage point to form a glycopeptide bond.

In a second aspect, the present application provides a nucleic acid molecule. The nucleic acid molecule includes a DNA fragment for encoding the antigen-binding fragment and/or the antibody according to the first aspect.

In a third aspect, the present application provides an expression vector. The expression vector includes the nucleic acid molecule according to the second aspect.

In a fourth aspect, the present application provides a host cell. The host cell includes the expression vector according to the third aspect, and/or a genome of the host cell is integrated with the nucleic acid molecule according to the second aspect.

In a fifth aspect, the present application provides a method for preparing an antibody. The method includes the following steps:
(1) ligating an encoding nucleic acid of the antibody according to the first aspect to a plasmid, transferring into a competent cell and culturing, then selecting monoclonal cells for screening;
(2) extracting an expression vector of a screened positive clone, transferring into a host cell, culturing and collecting a supernatant, then separating and purifying to obtain the antibody.

As a preferred technical solution, the present application provides a method for preparing an antibody. The method includes the following steps:
transfecting host cells transiently or stably with an antibody secretion system containing a heavy chain (HC) expression vector and a light chain (LC) expression vector in a specific ratio, or with a single vector containing coding HC and LC sequences, wherein the host cells may be, for example, HEK 293 or CHO;
obtaining purified antibody from a cell culture supernatant by a conventional method, for example, for Fab fragments, filtering the culture supernatant with a MabSelect column (GE Healthcare) or a KappaSelect column (GE Healthcare) equilibrated by phosphate buffer (pH=7.4), and removing non-specific binding components of the columns by washing; eluting the bound antibody with pH gradient eluent (20 mM pH=7 Tris buffer to 10 mM pH=3.0 sodium citrate buffer, or pH=7.4 phosphate buffer to 100 mM pH=3.0 glycine buffer);
detecting the antibody through SDS-PAGE, and optionally further purifying;
concentrating and/or sterile filtering the antibody to remove soluble aggregates and multimers with conventional techniques, wherein conventional techniques includes size-exclusion chromatography, hydrophobic interaction chromatography, ion exchange chromatography, multimodal chromatography or hydroxyapatite chromatography;
after the step of chromatographic concentration, the purity of the antibody is greater than 95%, and a product of the high-purity antibody is frozen or lyophilized at −70° C.

In a sixth aspect, the present application provides a pharmaceutical composition. The pharmaceutical composition includes the antibody according to the first aspect.

Preferably, the pharmaceutical composition further includes an anti-tumor drug.

Preferably, the pharmaceutical composition further includes any one or a combination of at least two of a pharmaceutically acceptable carrier, a diluent or an excipient.

In a seventh aspect, the present application provides a use of the antigen-binding fragment and/or the antibody according to the first aspect, the nucleic acid molecule according to the second aspect, the expression vector according to the third aspect, the host cell according to the fourth aspect or the pharmaceutical composition according to the sixth aspect for preparing a tumor detection reagent and/or a tumor treatment drug.

Preferably, the tumor includes a tumor that is positive for B7H3 expression. Preferably, the tumor includes any one or a combination of at least two of nervous system tumor, melanoma, lung cancer, head and neck cancer, colorectal cancer, pancreatic cancer, gastric cancer, kidney cancer, bladder cancer, prostate cancer, breast cancer, ovarian cancer or hepatocellular carcinoma.

The existing art has reported a diagnostic value of B7H3 in diseases such as primary liver cancer (Guo Haosu, Yang Yirong, Li Kai, He Kangli, Li Xingyue, Liu Hong; Diagnostic Significance of Serum CYFRA21-1 and sB7-H3 in Primary Liver Cancer[J]; China Journal of Modem Medicine.), esophageal squamous cell carcinoma (Sun Nan, Liu Xinbo, Cao Nana, Wang Ling; Expression and Clinical Significance of B7-H3 Gene in Peripheral Blood of Patients with Esophageal Squamous Cell Carcinoma[J]; Chinese Journal of Surgical Oncology; 2019 11(04): 247-250.), neuroblastoma (Liao Ru, Sun Xiaofei, Zhen Zijun, Wang Juan, Huang Dongsheng; Expression and Clinical Significance of B7H3 in Neuroblastoma Tissues[J]; Chinese Journal of Applied Clinical Pediatrics; 2019(11): 842-847.), colon cancer (Liang Qunying, Zhang Yuwen, Qiu Xiaodi; Expression of B7-H3 Protein in Colon Carcinoma and Clinical Pathological Significance[J]; Chinese Journal of Practical Medicine; 2018 45(15): 48-52.) and head and neck squamous cell carcinoma (Mao Liang, Fan Tengfei, Wu Lei, Yu Guangtao, Deng Weiwei, Chen Lei, Bu Linlin, Ma Sirui, Liu Bing, Bian Yansong, Ashok B Kulkami, Zhang Wenfeng, Sun Zhijun; Selective Blocking of B7-H3 in Head and Neck Squamous Cell Carcinoma B7-H3 can Enhance Anti-tumor Immune Effect by Reducing Myeloid-derived Suppressor Cells[C]; Chinese Stomatological Association; 2017: 139.), and the anti-B7H3 antibody has a therapeutic effect on tumors that are positive for B7H3 expression. The anti-B7H3 antibodies 26B6, 6F7, 2B8 and 23H1 of the present application each have a significant binding ability to B7H3 and a broad application prospect in the diagnosis and treatment of tumors that are positive for B7H3 expression.

Compared with the existing art, the present application has the beneficial effects described below.

(1) The anti-B7H3 antibodies 26B6, 6F7, 2B8 and 23H1 of the present application each have the significant binding ability to B7H3 and can bind not only purified or free B7H3 proteins but also B7H3 proteins on a cell surface.

(2) After the antibody of the present application is humanized, an affinity between the antibody and B7H3 is further improved.

(3) The antibody and the humanized antibody of the present application each have an important application prospect in the treatment of B7H3-positive tumors.

### BRIEF DESCRIPTION OF DRAWINGS

FIG. 1 is a structure diagram of human B7H3.

FIG. 2 illustrates that bindings of antibodies to B7H3 are detected through flow cytometry.

FIG. 3 illustrates that bindings of antibodies to B7H3 are detected through an enzyme-linked immunosorbent assay (ELISA).

FIG. 4 illustrates that bindings of humanized antibodies to B7H3 are detected through flow cytometry.

FIG. 5 illustrates suitable conditions for screening bindings of an antibody 1-scFv-hFc (hz26B6-scFv-hFc) through flow cytometry.

FIG. 6 illustrates suitable conditions for screening bindings of an antibody 2-scFv-hFc (hz2B8-scFv-hFc) through flow cytometry.

FIG. 7 is a binding target of an antibody 1-scFv-hFc.

FIG. 8 is a binding target of an antibody 2-scFv-hFc.

FIG. 9 illustrates that bindings of an antibody 1-scFv-hFc to B7H3 are detected through flow cytometry.

FIG. 10 illustrates that bindings of an antibody 2-scFv-hFc to B7H3 are detected through flow cytometry.

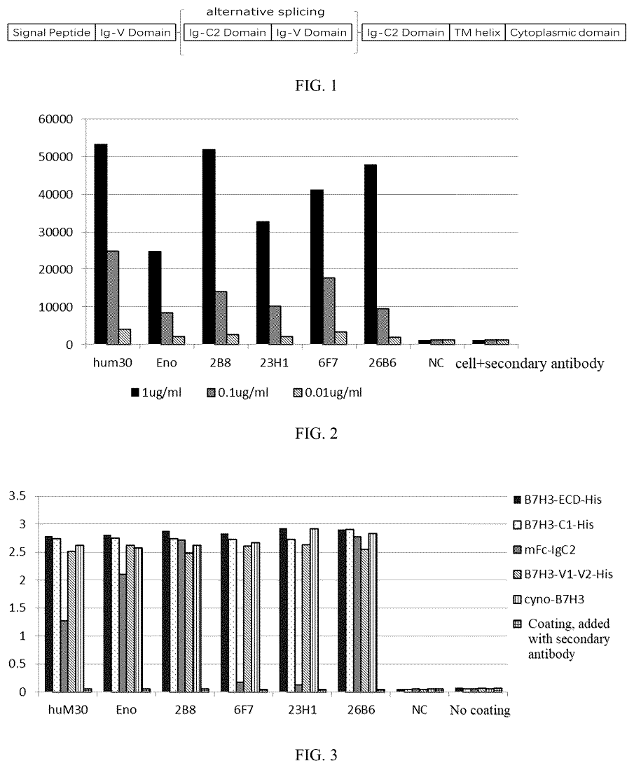

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions specified in the examples are conducted according to techniques or conditions described in the literature in the art or a product specification. The reagents or instruments used herein without manufacturers specified are conventional products commercially available from proper channels.

### Example 1 Expression and Purification of Antibodies

This example provides four anti-B7H3 antibodies 26B6 (SEQ ID NOs: 25 and 26), 6F7 (SEQ ID NOs: 29 and 30), 2B8 (SEQ ID NOs: 34 and 35) and 23H1 (SEQ ID NOs: 38 and 39). A method for preparing the antibodies includes the steps described below.

Stable transfection was performed on host cells HEK 293 using an antibody secretion system containing an HC expression vector and an LC expression vector in specific proportions. After a period of culture, a cell culture supernatant was filtered using a MabSelect column (GE Healthcare) equilibrated by phosphate buffer (pH=7.4), and a non-specific binding component of the column was removed by washing. The bound antibody was eluted with pH gradient eluent (20 mM pH=7 Tris buffer to 10 mM pH=3.0 sodium citrate buffer). The obtained eluent was detected through SDS-PAGE and further purified. The purified product was concentrated and sterile filtered to remove soluble aggregates and multimers. After the step of concentration, the purity of the antibody was greater than 95%, and the high-purity antibodies 26B6, 6F7, 2B8 and 23H1 were frozen or lyophilized at −70° C.

### Example 2 Affinity Test of Antibodies

Affinity detection was performed on the high-purity antibodies 26B6, 6F7, 2B8 and 23H1 prepared in Example 1 using a ForteBio affinity measurement method (P. Estep et al., High throughput solution-based measurement of antibody-antigen affinity and epitope binning. MAbs, 2013. 5(2): 270-278.), and control antibodies huM30 and Enoblituzumab (Eno) were set. huM30 is a humanized B7H3 antibody (CN103687945B) of Daiichi Sankyo Co., Ltd. in Japan, and DS-5573 is conducting a phase I clinical trial for the treatment of B7H3-positive solid tumors (NCT02192567). Enoblituzumab, a brand-new monoclonal antibody optimized by an immune molecule and aimed at a B7-H3 target, is developed by MacroGenics using an exclusive Fc optimization technology and has a unique antibody advantage and a therapeutic potential. With no such drug having been approved in the world, Enoblituzumab represents a leading B7-H3 antibody drug in the world.

Briefly, the antibody was loaded onto an AHQ sensor, and the sensor was equilibrated off-line in an assay buffer for 30 min and monitored online for 60 s for establishing a baseline. The antibody-loaded sensor was co-incubated with a 60 nM antigen B7H3 ECD-His for 5 min and transferred to the assay buffer, and a dissociation rate was measured after 5 min. Kinetic analysis was performed using a 1:1 binding model.

The results are shown in Table 1. Compared with the control antibodies huM30 and Eno on which clinical trials have been performed abroad, the 2B8, 26B8, 23H1 and 6F7 antibody screened in the present application have the same or better binding activity with human B7H3 ECD-his proteins.

TABLE 1
Affinities of binding antibodies to human B7H3 ECD-His

| | | Dissociation Equilibrium |
|---|---|---|
| Antigen | Antibody | Constant KD (M) |
| human B7H3 | huM30 | 3.46E−10 |
| ECD-His | Eno | 7.73E−10 |
| | 2B8 | 3.23E−10 |
| | 26B6 | 4.35E−10 |
| | 23H1 | 6.85E−10 |
| | 6F7 | 5.37E−10 |

### Example 3 Bindings of Antibodies to B7H3 on HEK293 Cells

In this example, the bindings of the antibodies prepared in Example 1 to B7H3 on the HEK293 cells were detected through flow cytometry. The steps are described below.

5×10⁵ HEK293 cells were resuspended with PBS+5% BSA and incubated for 30 min at 4° C. Different concentrations of antibody (1 μg/mL to 0.01 μg/mL, 10-fold gradient dilution) were added and incubated for 60 min at 4° C. After centrifugation and washing, a PBS+5% BSA solution containing an FITC-labeled secondary antibody (1:200, sigma, F9512) was added and incubated on ice for 30 min in the dark. Cells were washed three times before the flow cytometry analysis.

Control groups huM30, Eno, NC and Cell+secondary antibody were set.

The results are shown in FIG. 2. Using the flow cytometry method, the control antibodies (huM30, Eno) and the 2B8, 26B8, 23H1 and 6F7 antibody were tested for their binding abilities to HEK293 B7H3 proteins at a final concentration of 1 μg/mL, 0.1 μg/mL and 0.01 μg/mL, respectively. Compared with the control antibodies, the 2B8, 26B6, 23H1 and 6F7 antibody each have the same or better binding ability to the HEK293 B7H3 proteins.

### Example 4 ELISA Detection of Antibodies

Binding abilities of the antibodies 2B8, 26B8, 23H1 and 6F7 to extracellular fragments in different lengths of B7H3 proteins were detected through the indirect ELISA method. Five antigens (human B7H3-IgV-His, human B7H3-V1-C1-V2-His, human B7H3-IgC2, human B7H3-ECD-His and control proteins cyno-B7H3-His) were diluted to 0.2 μg/mL with a coating buffer, respectively, and added to a 96-well ELISA coating plate in 100 μL/well. After overnight incubation at 4° C., the wells were washed several times with a PBST solution and sealed with 1% BSA for 1 h at 37° C. 100 μL of the antibodies 2B8, 26B8, 23H1 and 6F7 with a final concentration of 0.5 μg/mL were added to each well, respectively, and incubated for 2 h at 37° C. After being washed five times with PBST, 100 μL of a diluted horseradish peroxidase (HRP)-labeled secondary antibody was added and incubated for 1 h at 37° C. After being washed five times with PBST, color development was performed with a chromogenic reagent for 20 min, and values were read on a microplate reader.

The results are shown in FIG. 3. The 2B8 and 26B6 antibody and the control antibodies huM30 and Eno have similar binding features of extracellular fragments of the B7H3 proteins, except that the 23H1 and 6F7 antibody mainly bind to sites other than Ig-C2 regions of the extracellular fragments of the B7H3 proteins.

### Example 5 Humanization of Antibodies

The antibodies 26B6, 6F7 and 2B8 prepared in Example 1 were humanized. Briefly, gene sequences of antibodies secreted by different hybridoma cells were compared with a gene sequence of a germline antibody from a human embryo to find a sequence with a high homology. An affinity of HLA-DR was analyzed and investigated, and a framework sequence of the germline from the human embryo with a low affinity was selected. Amino acid sequences of a variable region and a surround framework thereof were analyzed through molecular docking using a computer simulation technique. A spatial three-dimensional binding manner was investigated. Electrostatic force, van der Waals force, hydrophilicity, hydrophobicity and an entropy value were calculated, a key amino acid individual in the gene sequences of the antibodies secreted by each hybridoma cell was analyzed, which can interact with B7H3 and maintain a spatial framework. The key amino acid individual was grafted back to the selected gene framework of the germline from the human embryo, and based on this, an amino acid site of a framework region which must be retained was marked, a random primer was synthesized, a phage library was self-made, and a humanized antibody library was screened (A. Pini et al., Design and use of a phage display library: human antibodies with subnanomolar affinity against a marker of angiogenesis eluted from a two-dimensional gel. Journal of Biological Chemistry, 1998. 273(34): 21769-21776).

hz26B6 (SEQ ID Nos: 27 and 28), hz6F7 (SEQ ID NOs: 31 to 33) and hz2B8 (SEQ ID NOs: 36 and 37) were obtained.

### Example 6 Bindings of Humanized Antibodies to B7H3 on HEK293 Cells

In this example, the bindings of the humanized antibodies prepared in Example 5 to B7H3 on the HEK293 cells were detected through flow cytometry. The steps are described below.

2×10⁵ HEK293 cells were resuspended with PBS+5% BSA and incubated for 30 min at 4° C. Different concentrations of humanized antibody (5 μg/mL to 0.002286 μg/mL, 3-fold gradient dilution) were added and incubated for 60 min at 4° C. After centrifugation and washing, a PBS+5% BSA solution containing an FITC-labeled secondary antibody (1:200, sigma, F9512) was added and incubated on ice for 30 min in the dark. Cells were washed three times before the flow cytometry analysis. EC50 was calculated using GraphPad software.

Control groups huM30, Eno, NC and Cell+secondary antibody were set.

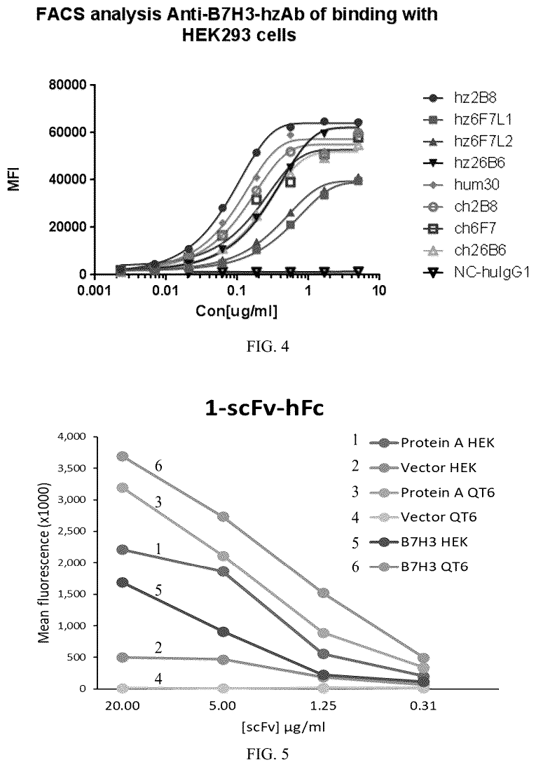

The results are shown in Table 2 and FIG. 4. hz26B6 was bound to B7H3 in a dose-dependent manner with an EC50 value (n=1) of 0.3063 μg/mL, hz2B8 was bound to B7H3 in a dose-dependent manner with an EC50 value (n=1) of 0.07789 μg/mL, hz6F7L1 was bound to B7H3 in a dose-dependent manner with an EC50 value (n=1) of 0.6038 μg/mL, hz6F7L2 was bound to B7H3 in a dose-dependent manner with an EC50 value (n=1) of 0.4096 μg/mL, and huM30 was bound to B7H3 in a dose-dependent manner with an EC50 value (n=1) of 0.09737 μg/mL. A humanized h2B8 antibody shows a better EC50 result than huM30 when bound to human B7H3.

In addition, light and heavy chain variable regions of murine 2B8, 26B6 and 6F7 antibodies were ligated to a constant region of a human antibody to obtain chimeric antibodies ch2B8, ch26B6 and ch6F7, where ch2B8 was bound to B7H3 in a dose-dependent manner with an EC50 value (n=1) of 0.1315 μg/mL, ch26B6 was bound to B7H3 in a dose-dependent manner with an EC50 value (n=1) of 0.2323 μg/mL, and ch6F7 was bound to B7H3 in a dose-dependent manner with an EC50 value (n=1) of 0.2244 μg/mL.

TABLE 2
Bindings of humanized antibodies to B7H3

| | h2B8 | h6F7H1L1 | h6F7H1L2 | h26B6 | huM30 | ch2B8 | ch6F7 | ch26B6 |
|---|---|---|---|---|---|---|---|---|
| EC50 | 0.07789 | 0.6038 | 0.4096 | 0.3063 | 0.09737 | 0.1315 | 0.2323 | 0.2244 |
| (μg/mL) | | | | | | | | |

### Example 7 Differences in Affinities Between Antibodies and B7H3 and Affinities Between Humanized Antibodies and B7H3

In this example, the differences in the affinities between the antibodies and B7H3 and the affinities between the humanized antibodies and B7H3 were detected using a ForteBio affinity measurement method.

The results are shown in Table 3. Compared with the results in Table 1, the humanized h2B8 antibody has a better KD value than the unmodified antibody and is better than the control antibody huM30.

TABLE 3
Affinities between humanized antibodies
and human B7H3 ECD-His

| Antigen | Antibody | KD (M) |
|---|---|---|
| human B7H3 | huM30 | 2.77E−10 |
| ECD-His | h2B8 | 2.59E−10 |
| | h26B6 | 7.53E−10 |
| | h6F7 | 1.01E−09 |
| | h6F7H1L1 | 3.70E−08 |
| | h6F7H1L2 | 1.42E−08 |

### Example 8 Membrane Protein Array (MAP) Verifies Binding Interactions Between Antibodies and B7H3

In this example, non-target binding interactions of the antibodies h26B6-scFv-hFc and h2B8-scFv-hFc were verified using the MAP. The steps are described below.

### (1) Determination of Screening Conditions

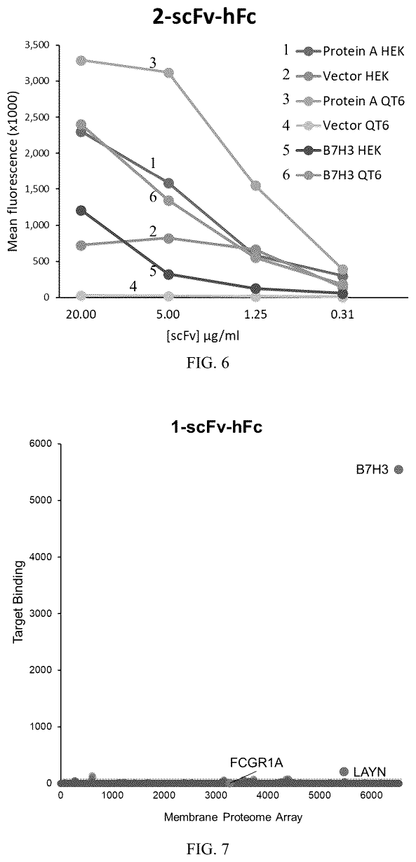

To optimize antibody detection conditions, HEK-293T cells and QT6 cells containing a plasmid containing a B7H3 antigen-encoding gene or an empty vector (pUC; negative control) were cultured in 384-well cell culture dishes. After culture for 24 h at 37° C. under 5% CO₂, each antibody to be detected was subjected to 4-fold dilution using a PBS solution containing 10% NGS, Ca²⁺ and Mg²⁺ and added in quadruplicate to transfected cells. The bindings of the antibodies to the cells were detected through high-throughput immunofluorescence flow cytometry (the conditions were the same as those described in Table 4). Dilution multiples of each antibody were determined using ForeCyt software (Intellicyt) according to a mean fluorescence intensity (MFI) value, and curves were plotted using Exce12017 (Microsoft) (as shown in FIGS. 5 and 6).

TABLE 4
Experimental parameters for screening membrane protein array

| | |
|---|---|
| 1-scFv-hFc | 2-scFc-hFc |

Experimental
Parameter

| Cells and Transfection | | | | |
|---|---|---|---|---|
| Type of Cells | HEK-293T, QT6 | | HEK-293T, QT6 | |
| Positive Control | Protein A, B7H3 | | Protein A, B7H3 | |
| Plasmid | | | | |
| Negative Control | pUC | | pUC | |
| Plasmid | | | | |
| Incubation | 36 h, 37° C., 5% CO₂ | | 36 h, 37° C., 5% CO₂ | |
| Condition | | | | |
| Blocking Buffer | 10% goat serum (Sigma G6767) | | 10% goat serum (Sigma G6767) | |
| for Primary | | | | |
| Antibody and | | | | |
| Secondary | | | | |
| Antibody | | | | |
| Primary Antibody | | | | |
| Name of | | | | |
| Antibody (Ab) | | | | |
| Initial | 1 | mg/ml | 1 | mg/ml |
| Concentration | | | | |
| Storage Condition | −20° C., 4° C. (after thawing) | | −20° C., 4° C. (after thawing) | |
| Lot Number | N/A | | N/A | |
| Validity Period | N/A | | N/A | |
| Source | Guangzhou Bio-gene Technology | | Guangzhou Bio-gene Technology | |
| | Co., Ltd. | | Co., Ltd. | |
| Concentration | 4-fold dilution (in quadruplicate, | | 4-fold dilution (in quadruplicate, | |
| | starting concentration 20 μg/ml) | | starting concentration 20 μg/ml) | |
| Incubation Time | 60 | min | 60 | min |
| (Room | | | | |
| Temperature) | | | | |
| Secondary Antibody | | | | |
| Target | Human IgG (Fc) | | Human IgG (Fc) | |
| Concentration | dilution in the buffer at 1:400 | | dilution in the buffer at 1:400 | |
| | (3.75 μg/ml) | | (3.75 μg/ml) | |
| Incubation Time | 30 | min | 30 | min |

| (Room | | |
|---|---|---|
| Temperature) | | |
| Manufacturer | Jackson ImmunoResearch | Jackson ImmunoResearch |
| Cat # | 109-606-008 | 109-606-008 |
| Antibody ID | AlexaFluor ® 647-AffiniPure Goat | AlexaFluor ® 647-AffiniPure Goat |
| | Fab Anti-Human IgG (Fc) | Fab Anti-Human IgG (Fc) |
| Washing | | |
| After Incubation | PBS (no Ca²⁺, Mg²⁺, Corning 46- | PBS (no Ca²⁺, Mg²⁺, Corning 46- |
| with Primary | 013-CM diluted in deionized | 013-CM diluted in deionized |
| Antibody | water) × 5 | water) × 5 |
| After Incubation | PBS (no Ca²⁺, Mg²⁺, Corning 46- | PBS (no Ca²⁺, Mg²⁺, Corning 46- |
| with Secondary | 013-CM diluted in deionized | 013-CM diluted in deionized |
| Antibody | water) × 2 | water) × 2 |
| Operator and Device | | |
| Liquid Treatment | PerkinElmer JANUS Automated | PerkinElmer JANUS Automated |
| | Workstation | Workstation |
| Flow Cytometer | Intellicyt iQue | Intellicyt iQue |
| Detection | laser 640 nm, filtration 675/25 | laser 640 nm, filtration 675/25 |
| Parameter | | |

### (2) Screening of the MAP

A plasmid containing cDNA clones of 5344 membrane proteins (containing more than 90% of human membrane protein groups) was introduced into the QT6 cells and cultured in a 384-well cell culture plate, a unique cDNA was contained in each well, and the cells were cultured at 37° C. under 5% CO₂. After culture for 36 h, the cells were stripped using a cell stripper and placed in rows and columns into a new 384-well plate of a two-dimensional model, and the plate was arranged using a JANUS automated workstation. Each well in the model plate contained 48 different over-expressed protein components, and each protein was represented by a unique combination of two different wells. The antibodies to be detected were added to an MAP detection template at a pre-measured concentration, washed with 1×PBS and detected using the flow cytometer (see Table 5). All data of the flow cytometer was subjected to result processing using the ForeCyt software (Intellicyt).

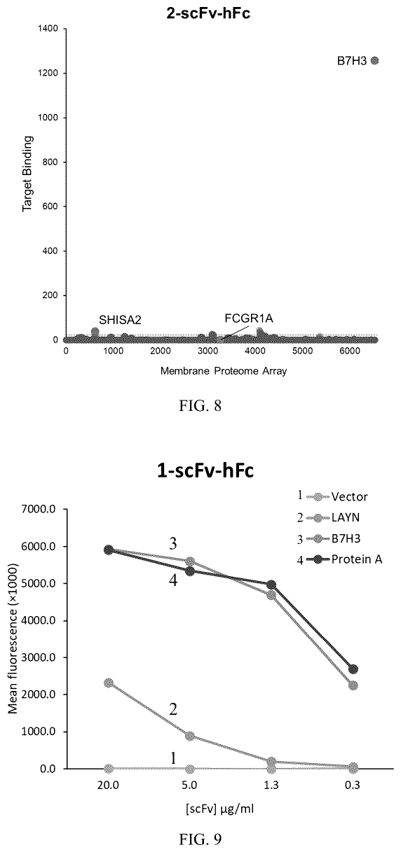

To obtain signal values of the bindings of the antibodies to be detected to each protein in the MAP, the two-dimensional binding data was converted using a standard method. Briefly, a value of each well (representing a separate overexpressed protein) was converted to a radian and plotted as a standard target binding curve using a formula r·sin (2θ). Excel2017 (Microsoft) was used for all values and analyses (as shown in FIGS. 7 and 8).

The results are shown in Table 6. 1-scFv-hFc binds to B7H3 and LAYN proteins on a cytoplasmic membrane, and 2-scFc-hFc binds to B7H3 and SHISA2 proteins on the cytoplasmic membrane.

TABLE 5
Experimental parameters for screening membrane protein array

| | |
|---|---|
| 1-scFv-hFc | 2-scFc-hFc |

Experimental
Parameter

| Membrane Protein Array (MAP) | | | | |
|---|---|---|---|---|
| Type of Cells | QT6 | | QT6 | |
| Incubation | 36 h, 37° C., 5% CO₂ | | 36 h, 37° C., 5% CO₂ | |
| Condition | | | | |
| Blocking buffer | 10% goat serum (Sigma G6767) | | 10% goat serum (Sigma G6767) | |
| for primary | | | | |
| antibody and | | | | |
| secondary | | | | |
| antibody | | | | |
| Primary Antibody | | | | |
| Name of | | | | |
| Antibody (Ab) | | | | |
| Initial | 1 | mg/ml | 1 | mg/ml |
| Concentration | | | | |
| Storage | −20° C., 4° C. (after thawing) | | −20° C., 4° C. (after thawing) | |
| Condition | | | | |
| Lot Number | N/A | | N/A | |
| Validity Period | N/A | | N/A | |
| Source | Guangzhou Bio-gene Technology | | Guangzhou Bio-gene Technology | |
| | Co., Ltd. | | Co., Ltd. | |
| Concentration | 20 | μg/ml | 5 | μg/ml |
| Incubation Time | 60 | min | 60 | min |
| (Room | | | | |
| Temperature) | | | | |
| Secondary Antibody | | | | |
| Target | Human IgG (Fc) | | Human IgG (Fc) | |
| Concentration | 1:400 (3.75 μg/ml) | | 1:400 (3.75 μg/ml) | |
| Incubation Time | 30 | min | 30 | min |

| (Room | | |
|---|---|---|
| Temperature) | | |
| Manufacturer | Jackson ImmunoResearch | Jackson ImmunoResearch |
| Cat # | 109-606-008 | 109-606-008 |
| Antibody ID | AlexaFluor ® 647-AffiniPure | AlexaFluor ® 647-AffiniPure |
| | Goat Fab Anti-Human IgG (Fc) | Goat Fab Anti-Human IgG (Fc) |
| Washing | | |
| After Incubation | PBS (no Ca²⁺, Mg²⁺, Corning 46- | PBS (no Ca²⁺, Mg²⁺, Corning 46- |
| with Primary | 013-CM diluted in deionized | 013-CM diluted in deionized |
| Antibody | water) × 5 | water) × 5 |
| After Incubation | PBS (no Ca2+, Mg²⁺, Corning 46- | PBS (no Ca²⁺, Mg²⁺, Corning 46- |
| with Secondary | 013-CM diluted in deionized | 013-CM diluted in deionized |
| Antibody | water) × 2 | water) × 2 |
| Operator and Device | | |
| Liquid Treatment | PerkinElmer JANUS Automated | PerkinElmer JANUS Automated |
| | Workstation | Workstation |
| Flow Cytometer | Intellicyt iQue | Intellicyt iQue |
| Detection | laser 640 nm, filtration 647/25 | laser 640 nm, filtration 647/25 |
| Parameter | | |

TABLE 6
Verified targets of membrane proteins

| Antibody | Target Gene (HGNC) | Protein Library |
|---|---|---|
| 1-scFv-hFc | B7H3 | Q5ZPR3 |
| (hz26B6-scFv-hFc) | LAYN | Q6UX15 |
| 2-scFv-hFc | B7H3 | Q5ZPR3 |
| (hz2B8-scFv-hFc) | SHISA2 | Q6UWI4 |

### (3) Re-Verification of the Bindings of the Antibodies to the Target

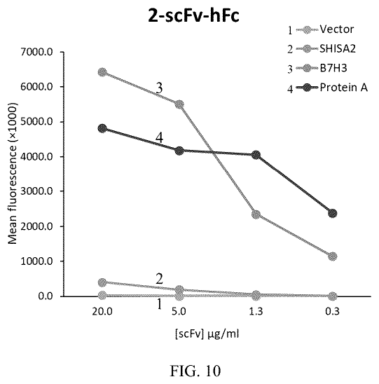

To verify the non-target binding interactions of the antibodies, plasmids encoding LAYN, SHISA2 and SEMA4B and an empty plasmid (pUC; negative control) were transfected into HEK 293T cells or QT6 cells and cultured in 384-well plates at 37° C. under 5% CO₂. After culture for 36 h, each antibody to be detected was subjected to 4-fold dilution and added to the transfected cells, and the bindings of the antibodies to the cells were detected using the high-throughput immunofluorescence flow cytometry (the conditions were the same as those described in Table 4). A dilution concentration of each antibody was measured using the ForeCyt software (Intellicyt), and a value of the dilution concentration was determined by an MFI value and plotted using Excel2017 (Microsoft) (as shown in FIGS. 9 and 10).

The results indicate that 1-scFv-hFc (hz26B6-scFv-hFc) and 2-scFv-hFc (hz2B8-scFv-hFc) can bind to B7H3 proteins without binding to other non-target proteins.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. An anti-B7H3 antigen-binding fragment, wherein an amino acid sequence of CDR3 of a heavy chain variable region of the antigen-binding fragment is as shown in SEQ ID NO: 1, SEQ ID NO: 2, SEQ ID NO: 3 or SEQ ID NO: 4;
an amino acid sequence of CDR3 of a light chain variable region of the antigen-binding fragment is as shown in SEQ ID NO: 5, SEQ ID NO: 6, SEQ ID NO: 7 or SEQ ID NO: 8.

2. The antigen-binding fragment according to claim 1, wherein an amino acid sequence of CDR2 of the heavy chain variable region of the antigen-binding fragment is as shown in SEQ ID NO: 9, SEQ ID NO: 10, SEQ ID NO: 11 or SEQ ID NO: 12;
preferably, an amino acid sequence of CDR2 of the light chain variable region of the antigen-binding fragment is as shown in SEQ ID NO: 13, SEQ ID NO: 14, SEQ ID NO: 15 or SEQ ID NO: 16;
preferably, an amino acid sequence of CDR1 of the heavy chain variable region of the antigen-binding fragment is as shown in SEQ ID NO: 17, SEQ ID NO: 18, SEQ ID NO: 19 or SEQ ID NO: 20;
preferably, an amino acid sequence of CDR1 of the light chain variable region of the antigen-binding fragment is as shown in SEQ ID NO: 21, SEQ ID NO: 22, SEQ ID NO: 23 or SEQ ID NO: 24.

3. The antigen-binding fragment according to claim 1, wherein the heavy chain variable region of the antigen-binding fragment comprises CDR1 as shown in SEQ ID NO: 17, CDR2 as shown in SEQ ID NO: 9 and CDR3 as shown in SEQ ID NO: 1;
the light chain variable region of the antigen-binding fragment comprises CDR1 as shown in SEQ ID NO: 21, CDR2 as shown in SEQ ID NO: 13 and CDR3 as shown in SEQ ID NO: 5;
preferably, the heavy chain variable region of the antigen-binding fragment comprises CDR1 as shown in SEQ ID NO: 18, CDR2 as shown in SEQ ID NO: 10 and CDR3 as shown in SEQ ID NO: 2;
the light chain variable region of the antigen-binding fragment comprises CDR1 as shown in SEQ ID NO: 22, CDR2 as shown in SEQ ID NO: 14 and CDR3 as shown in SEQ ID NO: 6;
preferably, the heavy chain variable region of the antigen-binding fragment comprises CDR1 as shown in SEQ ID NO: 19, CDR2 as shown in SEQ ID NO: 11 and CDR3 as shown in SEQ ID NO: 3;
the light chain variable region of the antigen-binding fragment comprises CDR1 as shown in SEQ ID NO: 23, CDR2 as shown in SEQ ID NO: 15 and CDR3 as shown in SEQ ID NO: 7;
preferably, the heavy chain variable region of the antigen-binding fragment comprises CDR1 as shown in SEQ ID NO: 20, CDR2 as shown in SEQ ID NO: 12 and CDR3 as shown in SEQ ID NO: 4; and
the light chain variable region of the antigen-binding fragment comprises CDR1 as shown in SEQ ID NO: 24, CDR2 as shown in SEQ ID NO: 16 and CDR3 as shown in SEQ ID NO: 8.

4. An anti-B7H3 antibody, comprising the antigen-binding fragment according to claim 1;
preferably, a heavy chain variable region of the antibody comprises an amino acid sequence as shown in SEQ ID NO: 25, and a light chain variable region of the antibody comprises an amino acid sequence as shown in SEQ ID NO: 26;
preferably, the heavy chain variable region of the antibody comprises an amino acid sequence as shown in SEQ ID NO: 27, and the light chain variable region of the antibody comprises an amino acid sequence as shown in SEQ ID NO: 28;
preferably, the heavy chain variable region of the antibody comprises an amino acid sequence as shown in SEQ ID NO: 29, and the light chain variable region of the antibody comprises an amino acid sequence as shown in SEQ ID NO: 30;
preferably, the heavy chain variable region of the antibody comprises an amino acid sequence as shown in SEQ ID NO: 31, and the light chain variable region of the antibody comprises an amino acid sequence as shown in SEQ ID NO: 32 or SEQ ID NO: 33;
preferably, the heavy chain variable region of the antibody comprises an amino acid sequence as shown in SEQ ID NO: 34, and the light chain variable region of the antibody comprises an amino acid sequence as shown in SEQ ID NO: 35;
preferably, the heavy chain variable region of the antibody comprises an amino acid sequence as shown in SEQ ID NO: 36, and the light chain variable region of the antibody comprises an amino acid sequence as shown in SEQ ID NO: 37;
preferably, the heavy chain variable region of the antibody comprises an amino acid sequence as shown in SEQ ID NO: 38, and the light chain variable region of the antibody comprises an amino acid sequence as shown in SEQ ID NO: 39;
preferably, the antibody further comprises a constant region;
preferably, the antibody is modified with a glycosylation group.

5. A nucleic acid molecule, comprising a DNA fragment for encoding the antigen-binding fragment according to claim 1.

6. An expression vector, comprising the nucleic acid molecule according to claim 5.

7. A host cell, comprising the expression vector according to claim 6.

8. A method for preparing an antibody, comprising the following steps:
(1) ligating an encoding nucleic acid of the antibody according to the first aspect to a plasmid, transferring into a competent cell and culturing, then selecting monoclonal cells for screening;
(2) extracting an expression vector of a screened positive clone, transferring into a host cell, culturing and collecting a supernatant, then separating and purifying to obtain the antibody.

9. A pharmaceutical composition, comprising the antibody according to claim 4;
preferably, the pharmaceutical composition further comprises an anti-tumor drug;
preferably, the pharmaceutical composition further comprises any one or a combination of at least two of a pharmaceutically acceptable carrier, a diluent or an excipient.

10. (canceled)

11. A method for preparing a tumor detection reagent and/or a tumor treatment drug using the antigen-binding fragment according to claim 1;
preferably, the tumor comprises a tumor that is positive for B7H3 expression;
preferably, the tumor comprises any one or a combination of at least two of nervous system tumor, melanoma, lung cancer, head and neck cancer, colorectal cancer, pancreatic cancer, gastric cancer, kidney cancer, bladder cancer, prostate cancer, breast cancer, ovarian cancer or hepatocellular carcinoma.


SEQUENCE LISTING (39 sequences)

SEQ ID NO: 1 - protein, 14 aa, artificial sequence
  CDR3
PYRDDGGFHWYFDV

SEQ ID NO: 2 - protein, 9 aa, artificial sequence
  CDR3
DSHYRPFAY

SEQ ID NO: 3 - protein, 13 aa, artificial sequence
  CDR3
GVFYYGYGAWFAY

SEQ ID NO: 4 - protein, 9 aa, artificial sequence
  CDR3
DSPYRPFAY

SEQ ID NO: 5 - protein, 9 aa, artificial sequence
  CDR3
QQWSSNPLT

SEQ ID NO: 6 - protein, 9 aa, artificial sequence
  CDR3
QNDYTYPLT

SEQ ID NO: 7 - protein, 9 aa, artificial sequence
  CDR3
QQHHEYPLT

SEQ ID NO: 8 - protein, 8 aa, artificial sequence
  CDR3
NDYTYPLT

SEQ ID NO: 9 - protein, 6 aa, artificial sequence
  CDR2
NPNTGG

SEQ ID NO: 10 - protein, 8 aa, artificial sequence
  CDR2
RNKANGYT

SEQ ID NO: 11 - protein, 6 aa, artificial sequence
  CDR2
NTNSGN

SEQ ID NO: 12 - protein, 8 aa, artificial sequence
  CDR2
RNKVNDYT

SEQ ID NO: 13 - protein, 7 aa, artificial sequence
  CDR2
DTSKLTS

SEQ ID NO: 14 - protein, 7 aa, artificial sequence
  CDR2
LASTRDS

SEQ ID NO: 15 - protein, 7 aa, artificial sequence
  CDR2
SGSTLQS

SEQ ID NO: 16 - protein, 7 aa, artificial sequence
  CDR2
LASTRES

SEQ ID NO: 17 - protein, 7 aa, artificial sequence
  CDR1
GYAFTEY

SEQ ID NO: 18 - protein, 7 aa, artificial sequence
  CDR1
GFTFTDY

SEQ ID NO: 19 - protein, 7 aa, artificial sequence
  CDR1
GYTFTDG

SEQ ID NO: 20 - protein, 7 aa, artificial sequence
  CDR1
GFTFTDY

SEQ ID NO: 21 - protein, 10 aa, artificial sequence
  CDR1
SASSSVSYMQ

SEQ ID NO: 22 - protein, 17 aa, artificial sequence
  CDR1
KSSQSLLNSGNQNNYLT

SEQ ID NO: 23 - protein, 11 aa, artificial sequence
  CDR1
RASKTISNYLA

SEQ ID NO: 24 - protein, 17 aa, artificial sequence
  CDR1
KSSQTLLNNGNQKNFLT

SEQ ID NO: 25 - protein, 125 aa, artificial sequence
  26B6HV
EVQLQQSGPELVKPGASVKISCKTSGYAFTEYTMHWVKQSQGKSLEWIGGINPNTGGTTYNQKFNGKATLTVDRSSSTAYMELRSLTSEDSAVYYCTRPYRDDGGFHWYFDVWGAGTAVTVSSAS

SEQ ID NO: 26 - protein, 112 aa, artificial sequence
  26B6LV
QIVLTQSPAVMSTSPGEKVTMTCSASSSVSYMQWYQQKSGTSPKRWIYDTSKLTSGVPARFSGSGSGTSYSLTISSMEAEDAATYYCQQWSSNPLTFGAGTKLELKRADAAP

SEQ ID NO: 27 - protein, 123 aa, artificial sequence
  hz26B6HV
EVQLVQSGAEVKKPGASVKVSCKASGYTFTEYTMHWVRQAPGQGLEWIGGINPNTGGTTYNQKFNGRVTMTRDTSISTAYMELSSLRSEDTAVYYCTRPYRDDGGFHWYFDVWGQGTLVTVSS

SEQ ID NO: 28 - protein, 109 aa, artificial sequence
  hz26B6LV
EIVLTQSPATLSLSPGERATLSCSASSSVSYMQWYQQKPGLAPRLLIYDTSKLTSGIPDRFSGSGSGTDFTLTISRLEPEDFAVYYCQQWSSNPLTFGGGTKVEIKRTV

SEQ ID NO: 29 - protein, 122 aa, artificial sequence
  6F7HV
EVKLVESGGGLVQPGGSLRLSCATSGFTFTDYYMSWVRQPPGKALEWLGFIRNKANGYTTEYSASVKGRFTISSDDSQSILYLQMNTLRAEDSATYYCARDSHYRPFAYWGQGTLVTVSAAS

SEQ ID NO: 30 - protein, 119 aa, artificial sequence
  6F7LV
DIQMTQSPSSLTVTAGEKVTMSCKSSQSLLNSGNQNNYLTWYQQKPGQPPKLLIYLASTRDSGVPDRFTGSGSGTDFTLTISSVQAEDLAVYYCQNDYTYPLTFGAGTKLELKRADAAP

SEQ ID NO: 31 - protein, 120 aa, artificial sequence
  hz6F7HV
EVQLVESGGGLVQPGGSLRLSCAASGFTFTDYYMSWVRQAPGKGLEWVAFIRNKANGYTTEYSASVKGRFTISRDDSKNSLYLQMNSLRAEDTAVYYCARDSHYRPFAYWGQGTLVTVSS

SEQ ID NO: 32 - protein, 116 aa, artificial sequence
  hz6F7LV1
DIVMTQSPLSLPVTPGEPASISCKSSQSLLNSGNQNNYLTWYLQKPGQSPQLLIYLASTRDSGVPDRFSGSGSGTDFTLKISRVEAEDVGVYYCQNDYTYPLTFGGGTKVEIKRTV

SEQ ID NO: 33 - protein, 116 aa, artificial sequence
  hz6F7LV2
DIVMTQSPDSLAVSLGERATINCKSSQSLLNSGNQNNYLTWYQQKPGQPPKLLIYLASTRDSGVPDRFSGSGSGTDFTLTISSLQAEDVAVYYCQNDYTYPLTFGGGTKVEIKRTV

SEQ ID NO: 34 - protein, 124 aa, artificial sequence
  2B8HV
QVQLQQSGPELVRPGVSVKISCKVSGYTFTDGAMHWVKRSHAKSLEWIGIINTNSGNTNYNQKFQGKATMTVDKSSSTAYMELARLTSEDSAIYYCARGVFYYGYGAWFAYWGQGTLVTVSAAS

SEQ ID NO: 35 - protein, 113 aa, artificial sequence
  2B8LV
DVQITQSPSYLTASPGETIIINCRASKTISNYLAWYQEKPGKTNKLLIYSGSTLQSGIPSRFSGSGSDTDFTLTISSLEPEDFAMYYCQQHHEYPLTFGAGTKLELKRADAAP

SEQ ID NO: 36 - protein, 122 aa, artificial sequence
  hz2B8HV
QVQLVQSGAEVKKPGASVKVSCKVSGYTFTDGAMHWVRQAPGQGLEWIGIINTNSGNTNYNQKFQGRVTMTRDTSISTAYMELSRLRSEDTAVYYCARGVFYYGYGAWFAYWGQGTLVTVSS

SEQ ID NO: 37 - protein, 110 aa, artificial sequence
  hz2B8LV
DIQLTQSPSFLSASVGDRVTINCRASKTISNYLAWYQQKPGKAPKLLIYSGSTLQSGVPSRFSGSGSGTDFTLTISSLQPEDFATYYCQQHHEYPLTFGGGTKVEIKRTV

SEQ ID NO: 38 - protein, 122 aa, artificial sequence
  23H1HV
EVKLVESGGGLVQPGGSLRLSCATSGFTFTDYYMSWVRQPPGKALEWLGFIRNKVNDYTTEYSVSVKGRFTISRDNSQTILYLQMNTLRAEDSATYYCARDSPYRPFAYWGQGTLVTVSAAS

SEQ ID NO: 39 - protein, 119 aa, artificial sequence
  23H1LV
DIVMTQSPSSLTVTAGENVTMSCKSSQTLLNNGNQKNFLTWYQQKPGQPPKLLIYLASTRESGVPDRFTGSGSGTDFTLTISSVQAEDLAVYYCQNDYTYPLTFGAGTKLELKRADAAP